(19) United States
(12) Patent Application Publication
(10) Pub. No.: US 2022/0082542 A1
(43) Pub. Date: Mar. 17, 2022

(54) SYSTEM FOR BEVERAGE ANALYSIS

(71) Applicants: ANYA MANISH LACHWANI, (US); MANISH KESHAV LACHWANI, (US); KAMINI MANOHARLAL JAGTIANI, (US)

(72) Inventors: ANYA MANISH LACHWANI, LOS ALTOS, CA (US); MANISH KESHAV LACHWANI, LOS ALTOS, CA (US); KAMINI MANOHARLAL JAGTIANI, LOS ALTOS, CA (US)

(21) Appl. No.: 17/018,638

(22) Filed: Sep. 11, 2020

(51) Int. Cl. G01N 33/14; A47J 31/58; G01D 21/02; A47G 19/22

(52) U.S. Cl. CPC G01N 33/14; A47G 19/2227; G01D 21/02; A47J 31/58

(57) ABSTRACT

Consumption of safe beverages improves health, physical performance, and prevents disease. A system includes a device that monitors a beverage within a vessel to determine if the beverage is safe to drink. The device may include a pH sensor to determine a pH value of the beverage. This pH value is compared to a pH range that is deemed safe. A comparison between the pH value and the pH range may be made, and the results presented to a user. The system may include a mixing device to automatically mix the beverage, or the system may prompt a user to manually mix the beverage. Data from other sensors may also be used to assess the beverage. The device may be implemented in various form factors such as a cup, lid, straw, stirrer, and so forth. The device may communicate with other devices, such as a phone, tablet, or server.

### BACKGROUND

Consumption of safe beverages improves health, physical performance, and prevents disease. For example, while performing a strenuous activity a person will become dehydrated if they do not replenish fluids lost due to respiration, perspiration, metabolic action, and so forth. However, the suitability of a beverage to be consumed may change. This may result in a beverage being suboptimal for consumption or even unsafe.

### BRIEF DESCRIPTION OF FIGURES

The detailed description is set forth with reference to the accompanying figures. In the figures, the left-most digit(s) of a reference number identifies the figure in which the reference number first appears. The use of the same reference numbers in different figures indicates similar or identical items or features. The figures are not necessarily drawn to scale, and in some figures, the proportions or other aspects may be exaggerated to facilitate comprehension of particular aspects.

FIG. 1 illustrates a system to analyze and advise a consumer as to the suitability for consumption of a beverage that is stored in a vessel, according to some implementations.

FIG. 2 is a block diagram of the components of the device, according to some implementations.

FIG. 3 illustrates the device that includes a vessel to hold a beverage and a removeable portion containing electronics and a mixer, according to one implementation.

FIG. 4 illustrates the device that includes a vessel that includes manual mixing features, the vessel to hold a beverage and a removeable portion containing electronics, according to one implementation.

FIG. 5 illustrates the device as a lid that may be affixed to a vessel to hold a beverage, according to one implementation.

FIG. 6 illustrates the device as a stirrer or insertable probe that may be placed at least partially within a vessel that holds a beverage, according to one implementation.

FIG. 7 illustrates the device that may be placed within a vessel and is immersed within a beverage, according to one implementation.

FIG. 8 illustrates user interfaces that may provide various functionality to the consumer, according to some implementations.

FIG. 9 is a flow diagram of a process for analyzing a beverage and providing output, according to some implementations.

While implementations are described herein by way of example, those skilled in the art will recognize that the implementations are not limited to the examples or figures described. It should be understood that the figures and detailed description thereto are not intended to limit implementations to the particular form disclosed but, on the contrary, the intention is to cover all modifications, equivalents, and alternatives falling within the spirit and scope as defined by the appended claims. The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean “including, but not limited to”.

### DETAILED DESCRIPTION

Beverages including water, electrolyte replacement drinks, energy drinks, and so forth have various physical and chemical characteristics. For example, a beverage has a particular pH, temperature, turbidity, and so forth. Characteristics of a beverage may change over time, as a result of changes in the surrounding environment, due to chemical or biological reactions within the beverage, chemical reactions with an interior surface of a vessel, and so forth. For example, a beverage may spoil due to the activity of microorganisms present in the beverage. In another example, the pH of a beverage may change and become more acidic due to chemical or biological reactions.

A beverage may be broadly considered to be safe or unsafe. A safe beverage may be further considered to be optimal or suboptimal for consumption. For example, a safe beverage would not cause harm to the consumer. In comparison, an unsafe beverage may cause harm to the consumer.

The safe beverage that is optimal may facilitate maintaining or improving health, physical performance, treatment or prevention of disease, and so forth. The safe beverage that is suboptimal does not offer these benefits. For example, a person suffering from severe dehydration and electrolyte imbalance may safely consume some water. However, water alone is suboptimal compared to a beverage that includes electrolytes, which may be optimal to improve rehydration. Continuing the example, a small amount of water may be better than no replenishing fluid, but too much water could result in hyponatremia and corresponding adverse health effects.

Traditional systems have focused on management of quantity of fluid consumed while ignoring actual and current characteristics of the fluids being consumed. For example, traditional systems may advise you to consume 2 liters of a fluid to maintain hydration, but are unable to determine a status as to whether the fluid you are consuming should even be consumed.

Described in this disclosure is a system that comprises a device with one or more sensors to measure one or more characteristics of a beverage. These characteristics may include, but are not limited to, pH (indicative of concentration of hydrogen ions), pO (indicative of concentration of hydroxide ions), temperature, level of fluid in a vessel, turbidity, and so forth. Values of the one or more characteristics are used to determine a status that is indicative of whether the beverage is safe or unsafe. If safe, the system may determine whether the beverage is optimal or suboptimal. In some implementations, the values used to make these determinations may be based on a selection of a type of beverage that is within the vessel. For example, a fruit juice may have a safe range that is more acidic than plain water.

Beverages may be subject to stratification due to gravity, environmental effects, and so forth. For example, a beverage may settle into layers as denser materials in suspension descend. The device may include a mixer that uses a motor to stir the beverage within the vessel. This mixing may be performed responsive to a value of a characteristic, at a predetermined time, and so forth. For example, if a first pH value exceeds a threshold value, a motor may be activated to stir the beverage. During or after the stirring, a second pH value may be determined and compared to the threshold value. Mixing may improve the accuracy of the measurement of the one or more characteristics measured by the sensors. Mixing may also improve safety of the consumer by preventing the consumer from inadvertently ingesting a stratified layer.

The device may include one or more output devices. For example, the device may include one or more output devices, such as a display, light emitting diodes, speaker, and so forth. Output may be presented to a consumer that indicates the status of the beverage. For example, a green light may indicate the beverage is safe and optimal, an amber light may indicate the beverage is safe but suboptimal, and a red light may indicate that the beverage is deemed unsafe.

The device may be implemented in various form factors, including but not limited to, cups, lids, straws, stirrers, water fountains, and so forth. The device may be used to facilitate consumption of beverages by any organism. For example, the device may be used by humans, pets, or other animals to facilitate safe consumption of beverages.

### Illustrative System

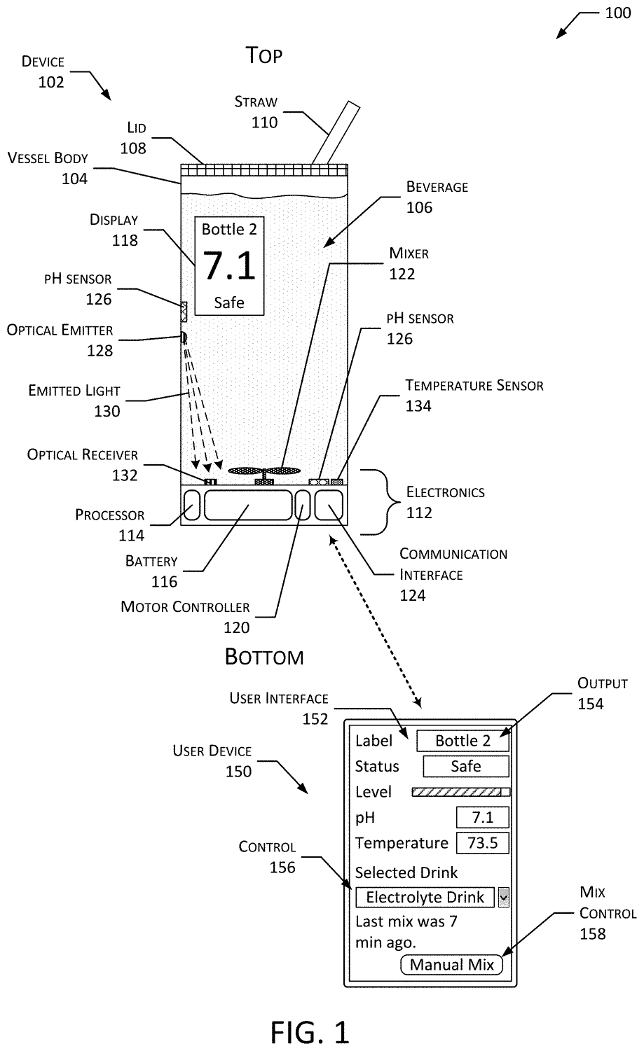

FIG. 1 illustrates a system 100 to analyze and advise a consumer as to the suitability of a beverage stored in a vessel to be consumed, according to some implementations.

The system 100 comprises a device 102. The device 102 may include a vessel body 104 that is configured to contain a beverage 106. The beverage 106 may include plain water, flavored water, electrolyte replacement drinks, energy drinks, soft drinks, brewed drinks, meal replacement liquids, and so forth. A lid 108 may be provided to prevent spillage, avoid ejection of a portion of the beverage 106 during operation of a mixer 122, and so forth. A straw 110 or other opening allows the beverage 106 to pass from within the vessel body 104 to a consumer for consumption. In some implementations the device 102 may utilize a rigid or non-rigid vessel to contain the beverage 106. For example, the beverage 106 may be contained within a non-rigid vessel comprising a flexible bladder.

The device 102 includes electronics 112. The electronics 112 may comprise a hardware processor (processor) 114, a battery 116, a display 118, a motor controller 120, a mixer 122, a communication interface 124, and one or more sensors. The processor 114 executes one or more instructions and may provide various functions such as using the one or more sensors to acquire sensor data, sending the sensor data using the communication interface 124, analyzing the sensor data, and so forth. The processor 114 may control operation of the motor controller 120. For example, the processor 114 may send a signal to the motor controller 120. Responsive to the signal, the motor controller 120 may operate a motor of the mixer 122. The motor may rotate a mixing element, such as a propeller, that causes movement of the beverage 106 within the vessel body 104. The mixing element may comprise a material that is designed to avoid harm in the event of an accidental contact with the consumer. For example, the mixing element may comprise a flexible plastic, elastomeric material, and so forth.

The processor 114 may operate the display 118 to provide output to the consumer. For example, the display 118 may be used to present a label associated with the device 102, such as “Bottle 2” and output indicative of one or more characteristics such as pH, status of the beverage such as “safe”, and so forth. In the implementation depicted here, the display 118 is positioned on the vessel body 104. For ease of illustration, and not as a limitation, electronic contacts, such as a flexible printed circuit, are not shown in these figures that connect the various devices to one another. Other information may also be presented by the display 118. For example, an identification of the type of beverage may be shown.

The one or more sensors of the device 102 may include one or more of a pH sensor 126, an optical emitter 128 and optical receiver 132, temperature sensor 134, and so forth.

The pH sensor 126 is configured to determine a pH value indicative of a concentration of hydrogen ions that are present within the beverage 106. For example, the pH sensor 126 may comprise a pH electrode arranged to come in contact with the beverage 106. The pH electrode may be connected to circuitry to determine output indicative of a pH value. For example, the circuitry may comprise a Texas Instruments LMP91200 by Texas Instruments Incorporated, of Dallas, Tex., United States of America. One or more pH sensors 126 may be arranged within the vessel body 104. For example, a first pH sensor 126 is shown on a bottom of the vessel while a second pH sensor 126 is shown approximately midway between the bottom and the top of the vessel body 104.

The optical emitter 128 and the optical receiver 132 may be used to determine turbidity of the beverage 106. Turbidity data comprises turbidity values that are indicative of cloudiness or haziness of a liquid that has some material suspended therein. For example, the optical emitter 128 may comprise a light emitting diode that generates emitted light 130 while the optical receiver 132 comprises a photodiode that is able to detect the emitted light 130. The processor 114 may operate the optical emitter 128 to generate emitted light 130 at one or more wavelengths, one or more intensities, and so forth. Output from the optical receiver 132 may be used to determine turbidity, color, and so forth of the beverage 106. In some implementations, data from the optical receiver 132 may be used to characterize a type of beverage within the vessel 104. In other implementations other optical techniques may be used to determine one or more characteristics of the beverage 106, including but not limited to polarimetry, spectroscopy, and so forth.

The temperature sensor 134 may include a thermocouple or other transducer. One or more temperature sensors 134 may provide sensor data indicative of temperature within the vessel body 104, temperature outside of the vessel body 104, temperature of the electronics 112, and so forth.

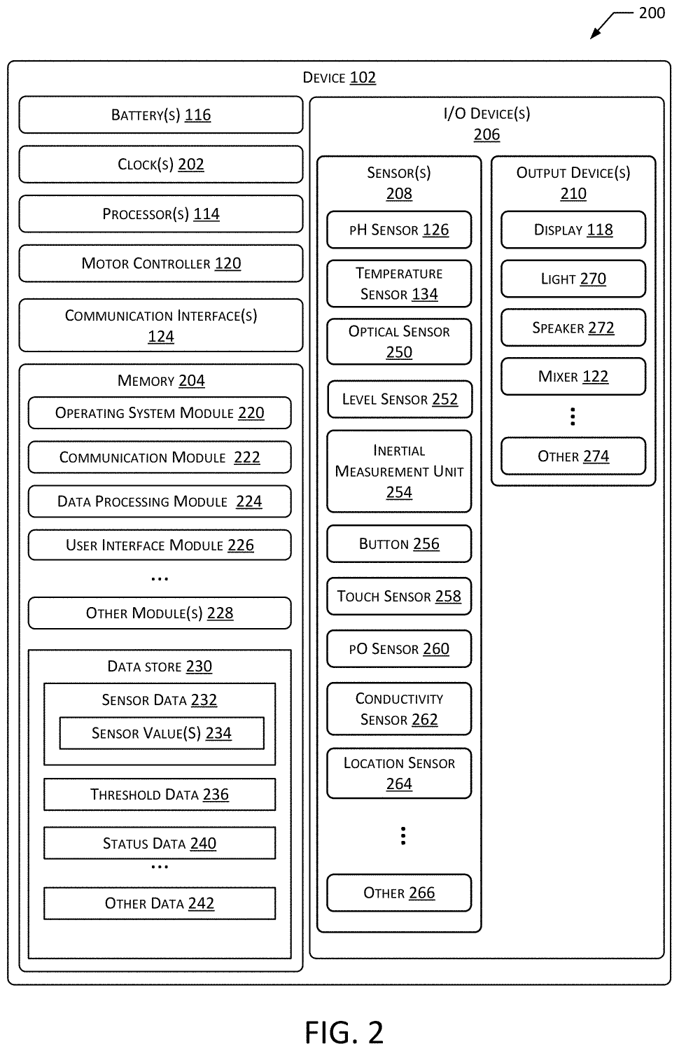

The components of the device, including the sensors, are discussed in more detail with regard to FIG. 2.

The device 102 may be in communication with an external device via the communication interface 124. For example, the external device may comprise one or more of a user device 150, server, and so forth. The user device 150 may comprise a wearable device, smartphone, tablet computer, desktop computer, in-vehicle system, fitness monitor, exercise equipment, and so forth. In this illustration, the user device 150 is in communication with the device 102. The user device 150 presents a user interface 152. The user interface 152 may comprise one or more outputs 154, controls 156, and so forth. The outputs 154 provide information for the consumer, while the controls 156 allow the user interface 152 to accept input from the consumer. In some implementations, a mix control 158 may be provided. Actuation of the mix control 158 may result in the processor 114 directing the motor controller 120 to operate the motor of the mixer 122 to mix the beverage 106.

In this illustration, the user interface 152 includes a label associated with the device, status of the beverage 106, level indicator that is indicative of a level of the beverage 106 in the vessel body 104, pH, and temperature. A control 156 allows the consumer to select the type of beverage that is within the vessel body 104. For example, the control 156 may allow the consumer to select a type of beverage from a list, such as “plain water”, “electrolyte drink”, “energy drink”, “tea”, “coffee”, “sweet drink”, “fruit drink”, and so forth. In some implementations, based on the input provided via one or more of the controls 156, the system may determine one or more ranges associated with one or more characteristic values. For example, a first range of pH values may be associated with a “safe” status of plain water, while a second range of pH values are associated with a “safe” status of a fruit drink.

FIG. 2 is a block diagram 200 of the device 102, according to some implementations. The components illustrated here are provided by way of illustration and not necessarily as a limitation. For example, the device 102 may utilize a subset of the particular network interfaces, sensors 208, or output devices 210 depicted here, or may utilize components not pictured.

The device 102 may include one or more batteries 116 or other power supply to provide electrical power suitable for operating the components in the device 102. In some implementations other devices may be used to provide electrical power to the device 102. For example, power may be provided by wireless power transfer, capacitors, fuel cells, storage flywheels, and so forth.

One or more clocks 202 may provide information indicative of date, time, ticks, and so forth. For example, the processor 114 may use data from the clock 202 to associate a particular time with an action, acquisition of sensor data, and so forth.

The device 102 may include one or more hardware processors 114 (processors) configured to execute one or more stored instructions. The processors 114 may comprise one or more cores. The processors 114 may include microcontrollers, systems on a chip, field programmable gate arrays, digital signal processors, graphic processing units, general processing units, and so forth.

The processor 114 may control operation of the motor controller 120.

The motor controller 120 may drive a motor of the mixer 122. For example, the motor controller 120 may comprise circuitry to drive a brushless direct current (BLDC) motor that is mechanically coupled to a mixing element, such as a propeller.

The device 102 may include one or more communication interfaces 124 such as input/output (I/O) interfaces, network interfaces, and so forth. The communication interfaces 124 enable the device 102, or components thereof, to communicate with other devices or components. The communication interfaces 124 may include one or more I/O interfaces. The I/O interfaces may comprise Inter-Integrated Circuit (I2C), Serial Peripheral Interface bus (SPI), Universal Serial Bus (USB) as promulgated by the USB Implementers Forum, RS-232, and so forth.

The I/O interface(s) may couple to one or more I/O devices 206. The I/O devices 206 may include input devices such as one or more sensors 208. The I/O devices 206 may also include output devices 210 such as one or more of the display 118, a light 270, a speaker 272, the mixer 122, and so forth. In some embodiments, the I/O devices 206 may be physically incorporated with the device 102 or may be externally placed.

The network interfaces may be configured to provide communications between the device 102 and other devices such as the user device 150, access points, and so forth. The network interfaces may include devices configured to couple to personal area networks (PANs), local area networks (LANs), wireless local area networks (WLANS), wide area networks (WANs), and so forth. For example, the network interfaces may include devices compatible with Ethernet, Wi-Fi, Bluetooth, Bluetooth Low Energy, ZigBee, LoRA, and so forth. In another example, the network interface may comprise a wireless wide area network (WWAN) interface or a wireless cellular data network interface. Continuing the example, the network interface may be compliant with at least a portion of the 4G, LTE, 5G, or other standards. The network interfaces may include other equipment to send or receive data using other wavelengths or phenomena. For example, the network interface may include an ultrasonic transceiver used to send data as ultrasonic sounds, a light system that communicates by modulating a light source such as a light-emitting diode, and so forth.

The device 102 may also include one or more busses or other internal communications hardware or software that allow for the transfer of data between the various modules and components of the device 102.

As shown in FIG. 2, the device 102 includes one or more memories 204. The memory 204 may comprise one or more non-transitory computer-readable storage media (CRSM). The CRSM may be any one or more of an electronic storage medium, a magnetic storage medium, an optical storage medium, a quantum storage medium, a mechanical computer storage medium, and so forth. The memory 204 provides storage of computer-readable instructions, data structures, program modules, and other data for the operation of the device 102. A few example functional modules are shown stored in the memory 204, although the same functionality may alternatively be implemented in hardware, firmware, or as a system on a chip (SoC).

The memory 204 may include at least one operating system (OS) module 220. The OS module 220 is configured to manage hardware resource devices such as the I/O interfaces, the I/O devices 206, the communication interfaces 124, and provide various services to applications or modules executing on the processors 114.

Also stored in the memory 204 may be a data store 230 and one or more of the following modules. These modules may be executed as foreground applications, background tasks, daemons, and so forth. The data store 230 may use a flat file, database, linked list, tree, executable code, script, or other data structure to store information. In some implementations, the data store 230 or a portion of the data store 230 may be distributed across one or more other devices.

A communication module 222 may be configured to establish communication with other devices, such as the user device 150, a server, and so forth. The communications may be authenticated, encrypted, and so forth.

A data processing module 224 may execute instructions to acquire sensor data 232 from the one or more sensors 208. The data processing modules 224 may perform one or more functions including, but not limited to, acquiring sensor data 232, processing sensor data 232, or determining status data 240.

The data processing module 224 may send instructions or signals to operate one or more sensors 208. For example, the data processing module 224 may poll the sensors 208 to send sensor data 232 to the data processing module 224. In another implementation the data processing module 224 may receive data from one or more sensors 208.

The data processing module 224 may process the sensor data 232 to determine sensor values 234. For example, the data processing module 224 may apply one or more noise filtering techniques, calibration processes, and so forth to raw values in the sensor data 232 to determine the sensor values 234. In one implementation, the data processing module 224 may calculate one or more sensor values 234 that are indicative of a minimum, maximum, average, moving average, standard deviation, and so forth.

The data processing module 224 may compare the sensor values 234 to threshold data 236 that is indicative of one or more threshold values to determine status data 240. For example, the threshold data 236 may indicate a pH range that is associated with a safe beverage. In some implementations the threshold data 236 may be determined based on input from a consumer or based on sensor data 232. For example, the consumer may use the user interface 152 to select the type of beverage being assessed by the device 102. Based on the type of beverage, particular threshold data 236 may be retrieved. In another example, based on sensor data 232 the type of beverage may be determined. Continuing the example, sensor data 232 provided by optical sensors such as the optical emitter 128 and the optical receiver 132 may be used to determine the type of beverage.

Based on the comparison of the sensor values 234 to the threshold data 236, the status data 240 is determined that is indicative of the status of the beverage 106. For example, the status data 240 may be indicative of whether the beverage 106 is “safe”, “optimal”, “suboptimal”, or “unsafe”. In other implementations the status data 240 may be indicative of “acidic”, “neutral”, or “alkaline”. The status data 240 may be presented as words, numbers, colors, visual indicia, audio output, and so forth. For example, the status data 240 may represent “safe”, “optimal”, “suboptimal”, or “unsafe” by different colors of light, icons on a display, sounds, and so forth.

The data processing module 224 may facilitate safe operation of the device 102. In one implementation, the data processing module 224 may only operate the motor controller 120 when the device 102 is determined to be in a safe condition. For example, a switch may be used to determine that the lid 108 is affixed to the vessel body 104 before allowing the mixer 122 to operate. This prevents an inadvertent dispersal of the beverage 106 as a result of the mixing action, and also prevents the consumer from inadvertently coming into physical contact with the mixer 122 while in operation.

The data processing module 224 may use data from one or more sensors 208 to facilitate operation of the mixer 122. For example, if the IMU 254 provides sensor data 232 indicating that the device 102 has been stationary for a threshold amount of time, the mixer 122 may be activated. In comparison, if the IMU 254 provides sensor data 232 indicating that the device 102 has been in motion that exceeds a threshold value, the mixer 122 may not be used as the mechanical motion may be deemed sufficient to provide mixing of the beverage 106.

Sensor data 240 from the IMU 254 may be used to determine if a manual mixing operation has been completed. In one implementation, a mixing notification may be presented using one or more of the output devices 210. The mixing notification may provide an indication that the vessel body 104 and the beverage 106 contained therein should be manually mixed, such by shaking. For example, the mixing notification may be responsive to a determination that the pH value of the beverage 106 is outside of a specified range of pH values. The mixing notification may be presented. For example, the mixing notification may comprise text, an icon, color, or other information presented by the display 118. In another example, the mixing notification may comprise illuminating a light 270 with a particular color, blink pattern, and so forth.

The IMU 254 is used to acquire sensor data 240 representative of the motion of the device 102, if any. A determination is made, using the sensor data 240 from the IMU 254, of one or more of a mixing time interval, or a mixing level. For example, the mixing time interval may be indicative of a total time elapsed during which the continuous movement of the device 102, as measured by the IMU 254, exceeded one or more threshold of acceleration or rotation. In another example, the mixing level may be indicative of how vigorously the device 102 was moved. Continuing the example, the mixing level may be indicative of a count of samples that exceeded a threshold minimum acceleration or rotation value.

The data processing module 224 may determine, based on the sensor data 240 from the IMU 254 or the data such as the mixing time interval or the mixing level, that the mixing was inadequate. For example a second mixing notification may be presented using an output devices 210, to encourage additional manual mixing.

The data processing module 224 determine if the device 102 is in use or in an orientation unsuitable for the mixer 122 to operate, and if so to avoid operating the mixer 122. In one implementation, sensor data 240 is acquired using the IMU 254. For example, the sensor data 240 may include information indicative of an acceleration due to gravity with respect to one or more axis. A tilt angle may be determined based on the sensor data 240. The tilt angle may be compared to a threshold value. If the tilt angle is greater than the threshold value, the device 102 may be deemed to be in an orientation unsuitable for operation of the mixer 122. For example, the device 102 may be deemed unsuitable for operation of the mixer 122 if upside down. Responsive to the determination that the tilt angle is greater than the threshold value, the data processing module 224 may direct the motor controller 120 to the mixer 122 if currently in operation, to prevent the mixer 122 from operating, or both.

The data processing module 224 may use other data 242 as well during operation. In one implementation, activity input data may be determined that is indicative of an activity or type of activity that is being performed or expected to be performed by the consumer. For example, the consumer may use the user interface 152 to provide input indicative of a particular type of activity such as “walking”, “running”, “cycling”, and so forth. The activity input data may be used to determine one or more of the threshold values of the threshold data 236.

The other modules 228 may provide other functionality. In some implementations, a prediction module may use one or more of the sensor values 234 or previously stored data to determine a prediction of one or more characteristics of the beverage 106 at a future time. For example, the prediction module may determine, based on a current pH at a first time, a previous pH at a second time, and a current temperature, that the beverage 106 will likely have a status of “unsafe” in 45 minutes. In some implementations the prediction may be based on previously stored data. For example, sensor values 234 and status data 240 for a beverage 106 at an earlier time may be stored and used to determine a prediction. The prediction may be based on data acquired using one or more devices 102. For example, the devices 102 may provide information such as type of beverage, sensor data 232, and status data 240 to a server. The server may use this information to generate predictive models for particular types of beverages, particular physical configurations of the device 102, and so forth. For example, a first device 102 having an insulated vessel body 104 may exhibit slower temperature changes in the beverage 106 due to ambient temperature, compared to a second device having an uninsulated vessel body 104. As a result, different predictive models may be provided for each of the physical configurations.

The one or more of the modules described herein may be combined, separated, or omitted in different implementations. For example, the processor 114 may comprise a microcontroller that has been programmed to perform the various functions described with respect to the communication module 222, the data processing module 224, or the user interface module 226, and may omit the operating system module 220.

The device 102 may include one or more of the following sensors 208. The sensors 208 depicted here are provided by way of illustration and not necessarily as a limitation. It is understood that other sensors 208 may be included or utilized by the device 102, while some sensors 208 may be omitted in some configurations.

The sensors 208 may include one or more pH sensors 126 to measure hydrogen ion concentration present in the beverage 106. As described above, the pH sensors 126 may comprise a pH electrode that is in contact with the beverage 106. The pH electrode may be connected to circuitry to determine sensor data 232 indicative of a pH value.

The temperature sensor 134 provides information indicative of temperature of one or more of the beverage 106, the surrounding ambient environment, the electronics 112, and so forth. For example, the temperature sensor 134 may provide sensor data 232 indicative of a temperature of the beverage 106.

One or more optical sensors 250 may provide sensor data 232 about the beverage 206. The optical sensors 250 may comprise the optical emitter 128 and the optical receiver 132. Information from the optical sensors 250 may be used to measure characteristics such as color, turbidity, concentration of one or more chemical compounds, presence of one or more chemical compounds, and so forth. For example, the optical sensors 250 may comprise a polarimeter that measures the effect of a liquid on polarized light to measure the sugar content in the beverage 106. In another example, the optical sensors 250 may comprise a spectrometer. Continuing the example, the optical sensors 250 may comprise a Raman spectroscope producing sensor data 232 that may be used to determine the type of beverage 106, presence of contaminants in the beverage 106, and so forth.

A level sensor 252 may be used to provide information as to quantity of beverage 106 that is within the vessel body 104, that has been removed from the vessel body 104, and so forth. In one implementation, the level sensor 252 may comprise a capacitive device that determines quantity based on changes in capacitance to one or more capacitive elements that are produced by the beverage 106 being nearby. In other implementations the level sensor 252 may utilize other mechanisms, such as an optical device to measure time of flight of light between the level sensor 252 and an interface between the beverage 106 and the atmosphere, a weight sensor, and so forth. In some implementations the level sensor 252 may comprise a flow sensor from which sensor data is integrated to determine the level of beverage 106 in the container. For example, the flow sensor may comprise an ultrasonic sensor that measures the quantity of beverage 106 entering and exiting the device 102. This data may be integrated to calculate net quantity of beverage 106 within the device 102.

An inertial measurement unit (IMU) 254 provides sensor data 232 indicative of movement. This movement may comprise rotations about one or more axes, accelerations with respect to one or more axes, and so forth. A gyroscope may provide information indicative of rotation of an object affixed thereto. For example, a gyroscope may generate sensor data 232 that is indicative of a change in rotation of the device 102. The gyroscope may comprise mechanical, optical, micro-electromechanical, or other devices.

An accelerometer provides information indicative of a direction and magnitude of an imposed acceleration. Data such as rate of change or determination of changes in direction, speed, and so forth may be determined using the accelerometer. The accelerometer may comprise mechanical, optical, micro-electromechanical, or other devices.

The IMU 254 may comprise a prepackaged solid-state device that includes multiple axis gyroscopes and multiple axis accelerometers.

A button 256 may accept input from the consumer. For example, the button 256 may be used to provide input that is used to select the type of beverage, reset a timer, activate the mixer 122, and so forth. In some implementations buttons 256 or switches may be used to provide information about the physical configuration of the device 102. For example, a switch may be used to determine when a removeable vessel body 104 is affixed to a portion that includes the electronics 112. The switches may be magnetic, optical, mechanical, capacitive, and so forth.

A touch sensor 258 detects the presence of an object, such as a touch of the consumer. The touch sensor 258 may be operated as a switch, or provide input such as with a touchpad allowing input along one or more axes. For example, the touch sensor 258 may comprise a capacitive touch sensor that provides touch data indicative of the touch. In another example, the touch sensor 258 may comprise a force sensitive resistor.

A pO sensor 260 determines a concentration of hydroxide ions in the beverage 106. A mathematical relationship between pH and pO may be used in some implementations to determine pH from a pO reading, or vice versa.

A conductivity sensor 262 determines an electrical conductivity of the beverage 106. For example, the conductivity sensor 262 may comprise a pair of electrodes connected to circuitry that is used to determine how well the beverage 106 conducts electricity. In some implementations, resistivity may be measured.

A location sensor 264 provides location data indicative of a location. The location sensor 264 may comprise an optical, radio, or other navigational system such as a global navigation satellite system (GNSS) receiver. For example, the device 102 may include a global position system (GPS) receiver. For indoor operation, the location sensor 264 may comprise indoor position systems, such as using Wi-Fi Positioning Systems (WPS). The location sensor 264 may provide location data that is indicative of a relative location, such as “living room” or an absolute location such as particular coordinates indicative of latitude and longitude, or displacement with respect to a predefined origin.

The device 102 may include one or more output devices 210. A display 118 may comprise one or more of a liquid crystal display, light emitting diode display, electrophoretic display, cholesteric liquid crystal display, interferometric display, and so forth. The display 118 may be used to present visible information such as graphics, pictures, text, and so forth. In some implementations, the display 118 may comprise a touchscreen that combines a touch sensor 258 and a display 118.

A light 270 may be used to provide output. For example, the light 270 may comprise a light emitting diode (LED), quantum dot, electroluminescent device, fluorescent device, incandescent bulb, and so forth. The light 270 may be capable of providing output with different colors. For example, the light 270 may comprise a multi-color LED that may be driven to provide different color outputs such as red, amber, green, and so forth.

A speaker 272 may be used to provide audio output. For example, different sounds may be associated with different states indicated by the status data 240. If the data processing module 224 determines the status data 240 indicates an “unsafe” status, the speaker 272 may present a warning sound. In other implementations, a buzzer or other device may be used to provide audio output.

The mixer 122 may be used as an output device 210. For example, the mixer 122 may be operated to mix the beverage 106. In another example, the mixer 122 may comprise a motor that is driven such that audio output is generated by the motor. For example, during initial startup of a BLDC motor, audible sound may be produced.

In some implementations the mixer 122 may be used as an input device. For example, the mixer 122 may be used to determine the viscosity of the beverage 106. Based on a known amount of power applied to the motor, and by monitoring operation of the motor such as actual revolutions per minute (RPM), the viscosity of the beverage 106 may be determined. For example, given a first power level applied, the motor would be expected to have an RPM of 200 in water. However, if the actual RPM is measured at 173, the data processing module 224 may determine that the beverage 106 is thicker than water due to the increased resistance slowing down the rotation. In another example, if the RPM exceeds an expected value, it may be determined that the mixer 122 is no longer fully submerged in the beverage 106. This may be due to orientation of the device 102, such as the device 102 being upside down. This may be due to the vessel body 104 being empty of beverage 106. In some implementations data from other sensors 208 may be used for disambiguation. For example, if the IMU 254 reports that the device 102 is upright, and the motor of the mixer 122 indicates an actual RPM that is consistent with the mixer 122 spinning in the air, rather than a liquid, the device 102 may be determined to be empty of beverage 106.

The output devices 210 may include other 274 devices as well. For example, the device 102 may include a haptic output device that provides output that produces particular touch sensations to the user. Continuing the example, a motor with an eccentric weight may be used to create a buzz or vibration.

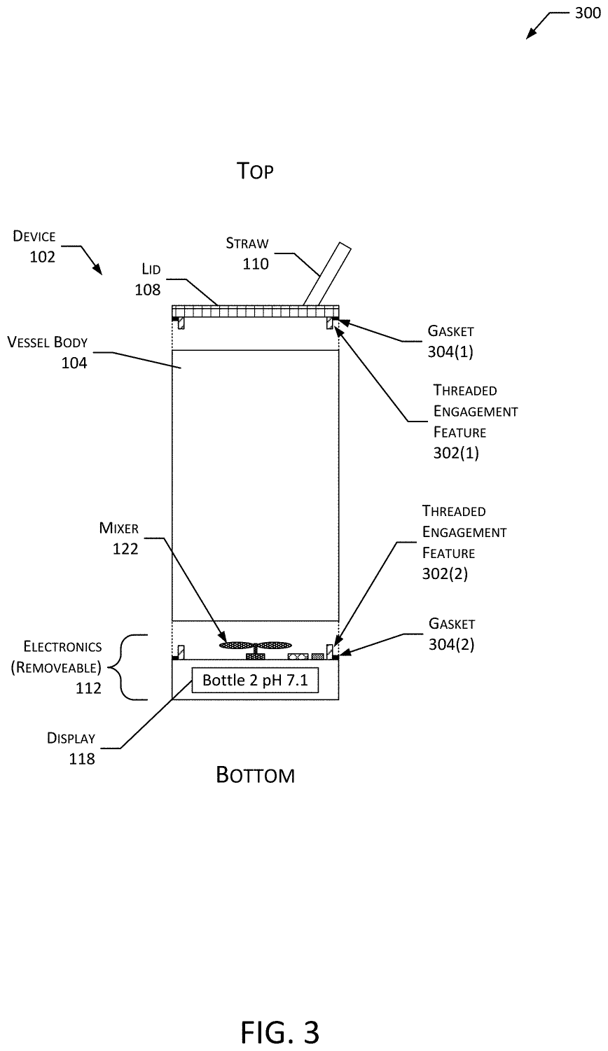

FIG. 3 illustrates at 300 an implementation of the device 102 that includes a vessel to hold a beverage 106 and includes a removeable portion containing electronics and a mixer, according to one implementation.

The device 102 may include one or more removeable components. These removeable components may be mechanically separated and joined. In one implementation, one or more threaded engagement features 302 may be used to provide mechanical engagement between two or more removeable components. In some implementations a gasket 304 may be provided to minimize leakage, prevent contaminants from entering the vessel body 104, and so forth. In other implementations other mechanisms may be used to join removeable components. For example, mechanical snap fit features, magnets, hinges, and so forth may be used.

In the implementation shown here, the lid 108 may screw onto the vessel body 104 using threaded engagement features 302(1) with a gasket 304(1) interposed between a portion of the vessel body 104 and a portion of the lid 108.

In the implementation shown here, the electronics 112 are also removeable from the vessel body 104. The portion containing the electronics 112 may include the mixer 122, display 118, and so forth.

The components of the device 102 may be separated to facilitate cleaning, maintenance, reconfiguration, and so forth. For example, the device 102 may be separated into the lid 108, vessel body 104, and electronics 112. The lid 108 and the vessel body 104 may be placed into a dishwasher for cleaning, while the electronics 112 are hand washed. The consumer may exchange components to provide desired functionality. For example, a first vessel body 104 with a capacity of 1 liter may be used for a marathon, while a second vessel body 104 with a capacity of 500 milliliters may be used for a shorter run or exercise at the gym. In yet another example, an insulated vessel body 104 may be used in a hot climate to minimize warming of a cold beverage 106, while an uninsulated vessel body 104 may be used during weight-sensitive activities such as mountain climbing.

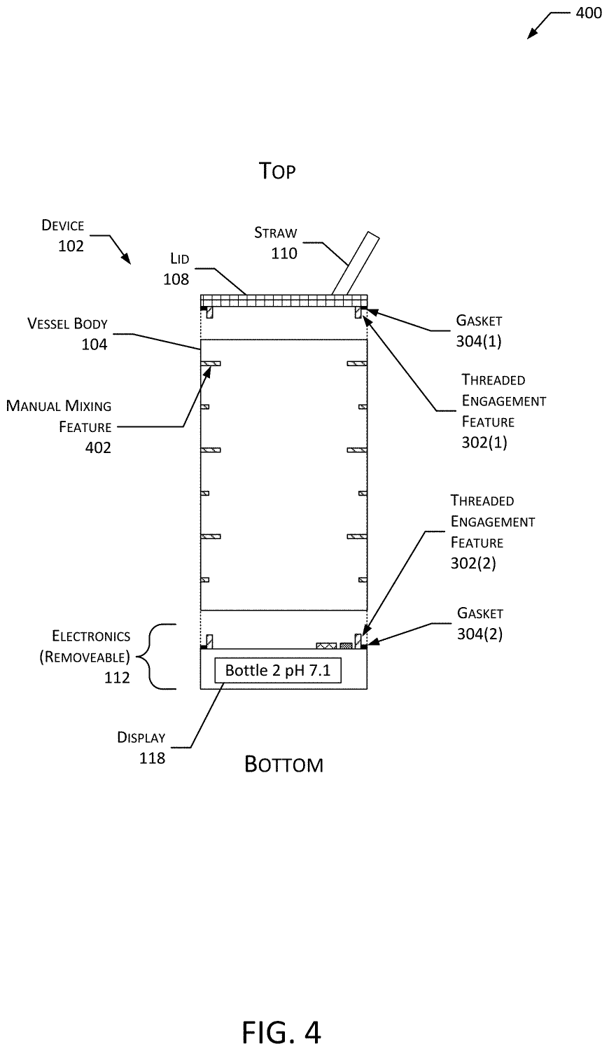

FIG. 4 illustrates at 400 an implementation of the device 102 that includes a vessel that includes manual mixing features, the vessel to hold a beverage 106 and a removeable portion containing electronics 112, according to one implementation.

In some implementations the mixer 122 may be omitted. The vessel body 104 or other component of the device 102 may include one or more manual mixing features 402. For example, the manual mixing features 402 may comprise protrusions that extend from an inner wall of the vessel body 104. The manual mixing features 402 may facilitate mixing by introducing turbulence in the beverage 106 under the influence of an external force. For example, the consumer may shake the device 102, or the device 102 may be moved as a result of being carried.

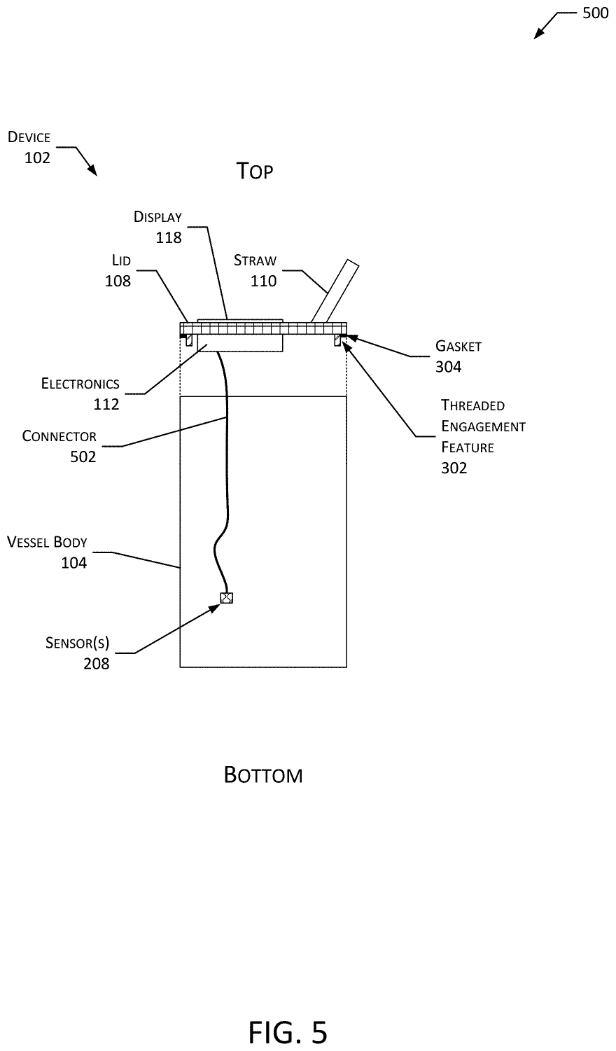

FIG. 5 illustrates at 500 an implementation of the device 102 as a lid 108 that may be affixed to a vessel to hold a beverage 106, according to one implementation. In this implementation, the electronics 112 are either attached to, or integrated into, the lid 108. The lid 108 may be affixed to a vessel body 104. Some of the one or more sensors 208 may be connected to the electronics 112 via a connector 502. For example, the pH electrodes used by the pH sensor 126 may be connected to the electronics 112 using the connector 502. During use, the lid 108 is affixed to the vessel body 104, and the sensors 208, at the end of the connector 502, are immersed in the beverage 106.

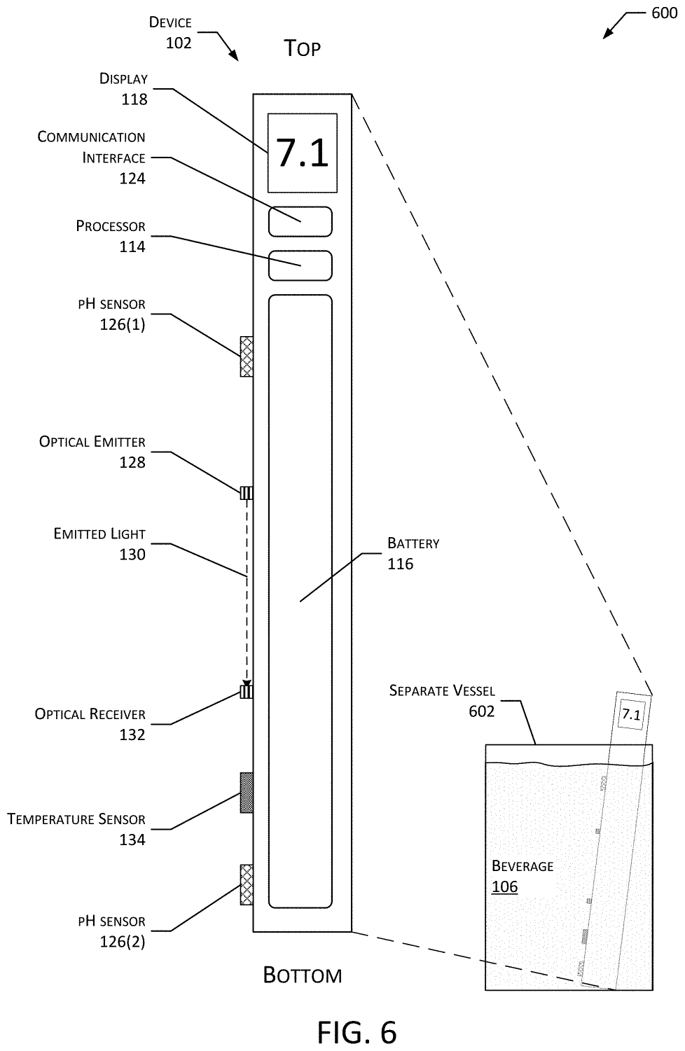

FIG. 6 illustrates at 600 an implementation of the device 102 as a stirrer or insertable probe that may be placed at least partially within a vessel that holds a beverage 106, according to one implementation.

In this implementation, the device 102 may be placed within a separate vessel 602 containing a beverage 106. For example, the device 102 may be inserted into a 5 gallon container of an electrolyte drink. In some implementations the device 102 may include a handle (not shown) or tether to facilitate handling.

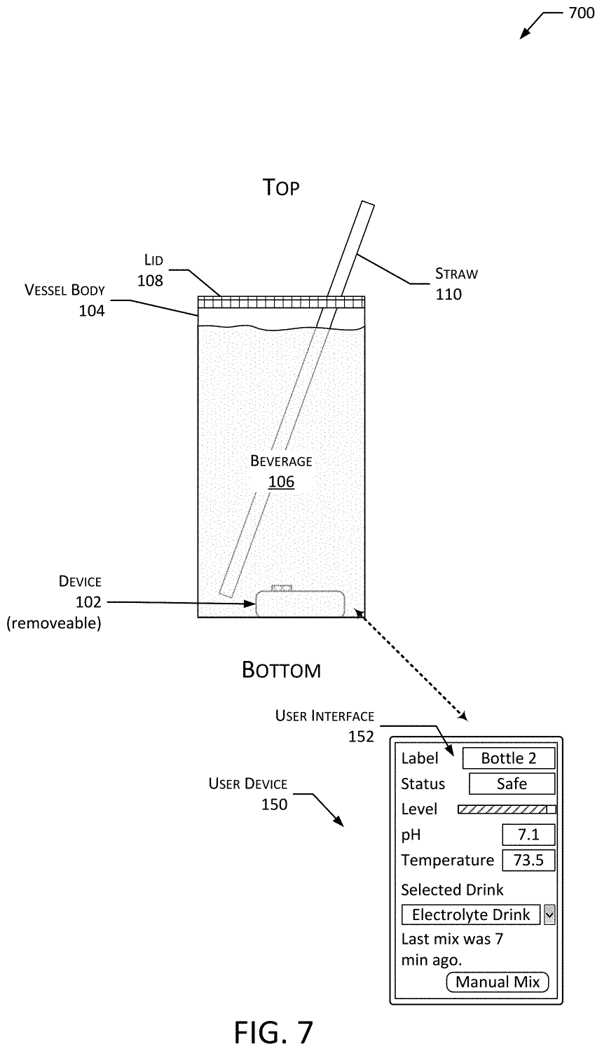

FIG. 7 illustrates at 700 an implementation of the device 102 that may be placed within a vessel and is immersed within a beverage 106, according to one implementation. In this implementation, the device 102 uses the communication interface 124 to communicate with an external device, such as the user device 150. The user device 150 may be used to inform the consumer as to the status of the beverage 106.

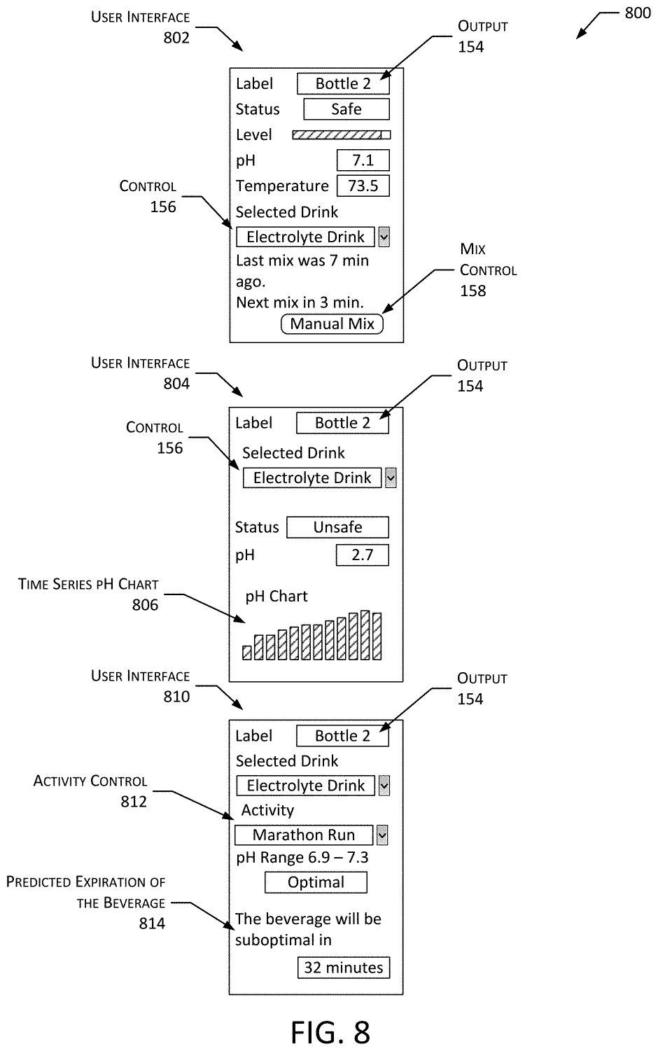

FIG. 8 illustrates at 800 several examples of user interfaces that may provide various functionality to the consumer, according to some implementations.

A first user interface 802 depicts various controls and output. In one implementation the consumer may specify the label associated with a particular device 102. For example, device 102(1) may be assigned the label “Bottle 2”, while device 102(2) may be assigned the label “Manish”.

The output includes a status indicator that is based on the status data 240. The status indicator may comprise visible output such as text, colors, light patterns, icons, and so forth. In another implementation the status indicator may comprise audio output, such as a particular sound. Also shown is the manual mix control 158. Actuation of the mix control 158 may initiate operation of the mixer 122, subject to safety or other operation considerations. For example, the manual mix control 158 may be inoperable if the lid 108 is not affixed to the vessel body 104.

A second user interface 804 depicts other output. For example, a time series pH chart 806 is shown, indicating the change of pH in the beverage 106 over time.

A third user interface 810 depicts an activity control 812. The activity control 812 may allow the consumer to specify an activity or category of activity that is being engaged in and provide activity input data. In some implementations, the activity input data may be used to determine the threshold data 236 that is used to determine the status data 240. For example, an activity input of “walking” may have an “optimal” pH range of 6.5 to 7.3 while an activity input of “marathon run” may have an “optimal” pH range of 6.9-7.3.

As described above, a prediction module may be used to predict a value of one or more characteristics of the beverage 106 at a future time. As illustrated in the third user interface 810, a predicted expiration of the beverage 814 is shown. For example, the beverage 106 is expected to be “suboptimal” in 32 minutes.

By using the information presented in the user interface, the consumer is able to quickly and easily determine the status of the beverage 106. The consumer may also be able to better manage their rate of consumption. For example, given that the beverage 106 is predicted to become suboptimal at a particular time, the consumer may choose to consume the beverage 106 before expiration.

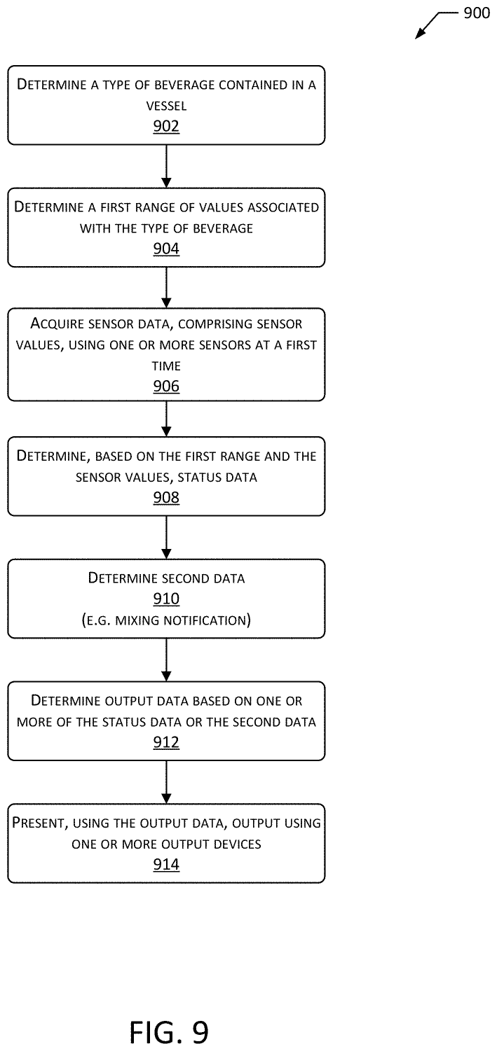

FIG. 9 is a flow diagram of a process for analyzing a beverage 106 and providing output, according to some implementations.

At 902, a type of beverage contained in the vessel is determined. In one implementation, the type of beverage may be received as user input from a user interface on a display. In another implementation, a default type of beverage may be assigned. In yet another implementation, the type of beverage may be determined based on sensor data 232 indicative of one or more characteristics of the beverage 106. For example, the beverage type may be inferred using data from the pH sensor 126 and the optical sensor 250.

At 904 a first range of values, such as pH values, that are associated with the type of beverage is determined. In some implementations, the first range may be stored as threshold data 236, and retrieved based at least in part on the type of beverage. The first range may be retrieved from an external source, entered by the consumer, and so forth.

The first range of values, such as pH values, may specify a range for which the particular type of beverage is deemed to be safe. For example, the first range for water may differ from a second range of fruit juice, which is more acidic.

In some implementations, the first range of values may be determined based at least in part on the activity input data. For example, the first range of pH values may differ for the same type of beverage, such as orange juice, depending on the activity being performed.

At 906, sensor data 232 is acquired using one or more sensors 208 at a first time. The sensor data 232 may include one or more sensor values 234, such as a first pH value acquired from a first pH sensor 126(1) or multiple pH values acquired by multiple pH sensors 126(N) at the same time or at a time interval associated with the first time. For example, a first pH sensor 126(1) may be located on a side wall of the vessel body 104, while a second pH sensor 126(2) may be located at or near the bottom of the vessel body 104.

At 908, status data 240 is determined based on the first range of values and the sensor values 234 determined at 906. The status data 240 is indicative of a status of the beverage 106 in the vessel body 104. For example, the status data 240 may indicate that the beverage 106 is “unsafe” for drinking when the pH value is outside the first range of values, or indicates “safe” to drink when the pH value is within the first range of pH values.

At 910, second data is determined based on one or more of the sensor data 232 or the status data 240. The second data may include one or more of a mixing notification or recommendation associated with the beverage 106, and so forth. For example, the mixing notification may indicate a time since last manual or automatic mixing of the beverage 106. In another example, the mixing notification may indicate a suggested next time to mix the beverage 106.

At 912 output data is determined based on one or more of the status data 240 or the second data. The output data includes data to be presented either on the vessel or an external device, or both. The output data may include a label associated with the beverage 106 or the vessel body 104, as well as the type of beverage 106 in the vessel body 104. The output data can also include a sensor value 234 such as a pH value of the beverage 106 and a status indicator that is based on the status data 240. Other sensor data can also be part of the output data, such as a temperature value or a level of the beverage 106.

The output data may also include the second data, such as one or more mixing notifications. For example, a notification to perform a manual mix of the beverage 106 may be presented using one or more output devices 210. Continuing the example, a flashing purple light may indicate that the consumer should perform a manual mix.

At 914, the output data is presented using one or more output devices 210. For example, the vessel body 104 or the electronics 112 portion of the device 102 may include a display 118 with text, lights, or sound. The output data can also be a display of an external device, such as a smartphone, tablet, wearable computing device, and so forth. The display may provide a user interface that includes some combination of text data, selectable controls, and graphical elements.

While the process is described with respect to pH values, the process may use one or more other sensor values from one or more of the sensors 208. In some implementations the status data 240 may be determined based on one or more of pH, temperature, optical characteristics, level, pO, conductivity, and so forth. For example, the status data 240 may be determined by comparing a pH value and a temperature value with predetermined ranges.

The processes and methods discussed in this disclosure may be implemented in hardware, software, or a combination thereof. In the context of software, the described operations represent computer-executable instructions stored on one or more computer-readable storage media that, when executed by one or more hardware processors, perform the recited operations. Generally, computer-executable instructions include routines, programs, objects, components, data structures, and the like that perform particular functions or implement particular abstract data types. Those having ordinary skill in the art will readily recognize that certain steps or operations illustrated in the figures above may be eliminated, combined, or performed in an alternate order. Any steps or operations may be performed serially or in parallel. Furthermore, the order in which the operations are described is not intended to be construed as a limitation.

Embodiments may be provided as a software program or computer program product including a non-transitory computer-readable storage medium having stored thereon instructions (in compressed or uncompressed form) that may be used to program a computer (or other electronic device) to perform processes or methods described herein. The computer-readable storage medium may be one or more of an electronic storage medium, a magnetic storage medium, an optical storage medium, a quantum storage medium, and so forth. For example, the computer-readable storage medium may include, but is not limited to, hard drives, optical disks, read-only memories (ROMs), random access memories (RAMs), erasable programmable ROMs (EPROMs), electrically erasable programmable ROMs (EEPROMs), flash memory, magnetic or optical cards, solid-state memory devices, or other types of physical media suitable for storing electronic instructions. Further embodiments may also be provided as a computer program product including a transitory machine-readable signal (in compressed or uncompressed form). Examples of transitory machine-readable signals, whether modulated using a carrier or unmodulated, include, but are not limited to, signals that a computer system or machine hosting or running a computer program can be configured to access, including signals transferred by one or more networks. For example, the transitory machine-readable signal may comprise transmission of software by the Internet.

Separate instances of these programs can be executed on or distributed across any number of separate computer systems. Thus, although certain steps have been described as being performed by certain devices, software programs, processes, or entities, this need not be the case, and a variety of alternative implementations will be understood by those having ordinary skill in the art.

Additionally, those having ordinary skill in the art will readily recognize that the techniques described above can be utilized in a variety of devices, physical spaces, and situations. Although the subject matter has been described in language specific to structural features or methodological acts, it is to be understood that the subject matter defined in the appended claims is not necessarily limited to the specific features or acts described. Rather, the specific features and acts are disclosed as illustrative forms of implementing the claims.

What is claimed is:

1. A system comprising:
a vessel body to hold a beverage; and
electronics coupled to the vessel body, the electronics comprising:
one or more first sensors to measure one or more first characteristics of the beverage, wherein the one or more first sensors comprise one or more pH sensors;
one or more output devices;
a battery;
a first communication interface;
a first memory storing first computer-executable instructions; and
one or more first hardware processors to execute the first computer-executable instructions to:
determine first data indicative of a type of beverage in the vessel body;
determine second data indicative of a pH range associated with the type of beverage;
acquire sensor data using the one or more pH sensors, wherein the sensor data comprises a first pH value of the beverage at a first time;
determine status data based on comparison of the first pH value and at least a portion of the second data; and
determine output data based at least in part on one or more of: the first data, the second data, the sensor data, or the status data.

2. The system of claim 1, the one or more first hardware processors to further execute the first computer-executable instructions to:
present the output data using the one or more output devices, the output data comprising one or more of:
an identification of the beverage or the vessel body,
an indication of a pH value of the type of beverage,
the status data, or
a mixing notification.

3. The system of claim 1, further comprising:
the one or more first hardware processors to further execute the first computer-executable instructions to:
transmit the output data to an external device using the first communication interface; and
the external device comprising:
a display;
a second communication interface;
a second memory storing second computer-executable instructions; and
one or more second hardware processors configured to execute the second computer-executable instructions to:
receive the output data using the second communication interface, wherein the output data comprises one or more of:
a label associated with the vessel body,
a status indicator associated with a status of the beverage,
a level indicator associated with a level of the beverage,
a pH value associated with the beverage,
a temperature associated with the beverage,
an indication of a type of beverage, or
a mixing notification; and
present the output data using the display.

4. The system of claim 1, wherein the electronics are removeable from the vessel body.

5. The system of claim 1, further comprising a motor controller and a mixer; and the one or more first hardware processors to further execute the first computer-executable instructions to:
determine one or more of the following:
the status data indicates a pH value of the beverage is outside the pH range specified by the second data, or
a time interval has expired; and
initiate operation of the mixer using the motor controller.

6. The system of claim 1, further comprising:
an inertial measurement unit; and
the one or more first hardware processors to further execute the first computer-executable instructions to:
present a mixing notification using the one or more output devices;
acquire second sensor data using the inertial measurement unit;
determine, using the second sensor data, one or more of:
a mixing time interval, or
a mixing level;
present a second mixing notification using the one or more output devices responsive to determining one or more of:
the mixing time interval being below a first threshold value, or
the mixing level being below a second threshold value.

7. The system of claim 1, the one or more first hardware processors to further execute the first computer-executable instructions to:
determine one or more mixing notifications based on the status data and the type of beverage;
wherein the output data comprises the one or more mixing notifications; and
send the output data to an external device using the first communication interface.

8. The system of claim 1, further comprising:
the one or more first hardware processors to further execute the first computer-executable instructions to:
transmit the output data to an external device using the first communication interface; and
the external device comprising:
a second communication interface;
a second memory storing second computer-executable instructions; and
one or more second hardware processors to further execute the second computer-executable instructions to:
receive the output data using the second communication interface;
determine a first time interval associated with a last mix of the beverage in the vessel body;
determine a second time interval associated with a next mixing of the beverage; and
determine a mixing notification.

9. The system of claim 1, the one or more output devices comprising one or more of:
one or more light sources,
one or more displays, or
one or more speakers; and
further comprising one or more of:
one or more temperature sensors,
one or more level sensors,
one or more optical sensors,
one or more inertial measurement units,
one or more buttons,
one or more touch sensors,
one or more pO sensors,
one or more conductivity sensors, or
one or more location sensors.

10. The system of claim 1, further comprising:
an inertial measurement unit;
a motor controller and a mixer; and
the one or more first hardware processors to further execute the first-computer executable instructions to:
acquire second sensor data using the inertial measurement unit;
determine, based on the second sensor data, a tilt angle;
responsive to determining the tilt angle is greater than a threshold value, performing one or more of:
stopping the mixer, if the mixer is operating, or
preventing the mixer from operating.

11. A method comprising:
determining a first range of pH values associated with a beverage in a vessel body;
acquiring, using at least one pH sensor, a first pH value of the beverage in the vessel body at a first time;
comparing the first pH value and the first range of pH values;
determining status data based on the comparing the first pH value and the first range of pH values;
determining output data based on one or more of:
the first range,
the first pH value, or
the status data; and
presenting at least a portion of the output data using one or more output devices.

12. The method of claim 11, further comprising:
determining a time interval remaining before a pH value of the type of beverage reaches a second pH value; and
wherein the output data is based on one or more of:
the time interval, or
a mixing notification based on the time interval.

13. The method of claim 11, further comprising:
operating, at a second time, a mixer associated with the vessel body;
acquiring, using the at least one pH sensor, a second pH value of the beverage at a third time after the second time;
comparing the second pH value and the first range of pH values;
determining second status data of the beverage; and
wherein the output data is based on one or more of:
the second pH value, or
the second status data.

14. The method of claim 11, further comprising:
receiving the output data at an external device; and
wherein the presenting at least a portion of the output data is performed at least in part by the external device.

15. A system comprising:
a vessel body to hold a beverage; and
electronics coupled to the vessel body, the electronics comprising:
one or more sensors;
a power supply;
a first communication interface;
a first memory storing first computer-executable instructions; and
one or more first hardware processors to execute the first computer-executable instructions to:
acquire, using the one or more sensors, sensor data associated with the beverage; and
send the sensor data to an external device.

16. The system of claim 15, wherein:
the sensor data comprises one or more of:
a pH value associated with the beverage in the vessel body,
a level of the beverage in the vessel body,
a temperature of the beverage in the vessel body,
turbidity data associated with the beverage,
motion data associated with motion of the beverage or the vessel body, touch data,
a pO level,
a conductivity of the beverage, or
location data indicative of a location of at least a portion of the system.

17. The system of claim 15, wherein:
the one or more sensors comprising one or more of:
a pH sensor,
a temperature sensor,
a level sensor,
an optical sensor,
a motion sensor,
a button,
a touch sensor,
a pO sensor,
a conductivity sensor, or
a location sensor.

18. The system of claim 15, further comprising:
one or more output devices, the one or more output devices comprising one or more of:
a display,
one or more lights, or
one or more speakers; and
the one or more first hardware processors to further execute the first computer-executable instructions to:
determine output data based on the second data; and
present at least a portion of the output data, using one or more of the one or more output devices.

19. The system of claim 15, further comprising:
a user device comprising:
a second communication interface;
an output device;
one or more second hardware processors configured to execute second computer-executable instructions to:
receive the second data using the second communication interface;
determine output data based at least in part on the second data, wherein the output data comprises one or more of:
a label associated with the beverage or the vessel body,
a status indicator,
a level indicator,
a pH value indicator,
a graphical element associated with one or more pH values,
a temperature indicator,
an indication of a predicted expiration of the beverage,
a first selectable control for selecting an activity or type of activity,
a second selectable control for selecting the type of beverage in the vessel body, or
a third selectable control associated with a mixing of the beverage in the vessel body; and
present the output data using the output device.

20. The system of claim 15, further comprising:
a mixer to mix a beverage in the vessel body;
the one or more first hardware processors to further execute the first computer-executable instructions to:
operate the mixer.
